(19) United States
(12) Patent Application Publication
(10) Pub. No.: US 2006/0270973 A1
(43) Pub. Date: Nov. 30, 2006

(54) Air bag nursing bra

(76) Inventor: Yun Yuan Chu, Taipei (TW)

Correspondence Address: Connolly Bove Lodge & Hutz, LLP, P.O. Box 2207, Wilmington, DE, 19899-2207, US

(21) Appl. No.: 11/136,107

(22) Filed: May 24, 2005

(51) Int. Cl. A61M 1/06

(52) U.S. Cl. 604/74

(57) ABSTRACT

Disclosed is a bra with air bags for massaging breast and draining breast milk. The air bags are inhaled and exhausted with a warm pulse positive pressure air to procure a warm massage effect for breasts, and further the air bag gradually squeezes the breasts from bottom of the breasts to nipple for draining breast milk and collecting breast milk into a baby bottle.

### BACKGROUND OF THE INVENTION

1. Field of the Invention

The present invention relates to a bra, and more particularly to an air bag nursing bra.

2. Description of the Prior Art

Recently, the breast pump is to drain the breast milk from breasts by creating vacuum to strongly suck local area of the nipple surrounding. As everyone knows, the breast milk is perfect and nature baby food. However, a career woman, during mammal period often, is unable to breast-feed in person because of job. The career woman has to get up to breast-feed at nigh even after coming off work.

Medical science verifies that the unpolluted breast milk can be frozen to preserve for several weeks. The frozen breast milk is warmed up with a pan of warm water for feeding baby. That is the reason why many breast pumps are available on the market. The principle of the conventional breast pumps is to cover the area of the nipple locally by a sucking disk. Then, an air pump of the breast pump is automatically or manually turned on to generate suction for draining breast milk. It discomforts woman on nipple eversion, hypertrophy, and darkened even. Furthermore, the woman has to prepare the breast with a hot compress and massage for promoting mammary gland lactation before draining breast milk by the breast bump. Moreover, they can not do anything due to operating breast bumps request one or full hands. In addition, the breast pump is hard to clean since it is integrated with the sucking disk and air pump. This may causes the breast milk to be polluted.

### SUMMARY OF THE INVENTION

In those conventional arts, the breast pump meets the problems mentioned above. In view of this, the present invention employs an air bag nursing bra to collecting breast milk. The bra comprises an air bag bra, an air pump, and a milk drainage assembly. The air pump provides a warm pressurized air and a negative pressure air for draining, and the milk drainage assembly is provided to drain the breast milk.

A user could clean the breasts before using the air bag nursing bra of the present invention, and then wears the bra according to an user's manual. Next, the user presses the automatically sucking process button which is on an operation panel, and a control circuit will automatically perform processes of warm massage and collecting breast milk. The positive/negative air pressure, inhalation/exhaust period, and the temperature of the bra can be adjusted through the control circuit. The user collects breast milk under the most comfortable setup at this point. Moreover, the user also can do thing in the range of the tube length.

### BRIEF DESCRIPTION OF THE DRAWINGS

FIG. 1 is schematic assembly diagram of the bra of the present invention;

FIG. 2 is a perspective view of the air bag bra of the present invention;

FIG. 3 is a schematic diagram of the air bag bra before inhaling;

FIG. 4 is a schematic diagram of the inhaled air bag bra squeezing the breasts;

FIG. 5 is a block diagram of the air pump of the present invention; and

FIG. 6 is schematic assembly diagram of the milk drainage assembly of the present invention

### DESCRIPTION OF THE PREFERRED EMBODIMENT

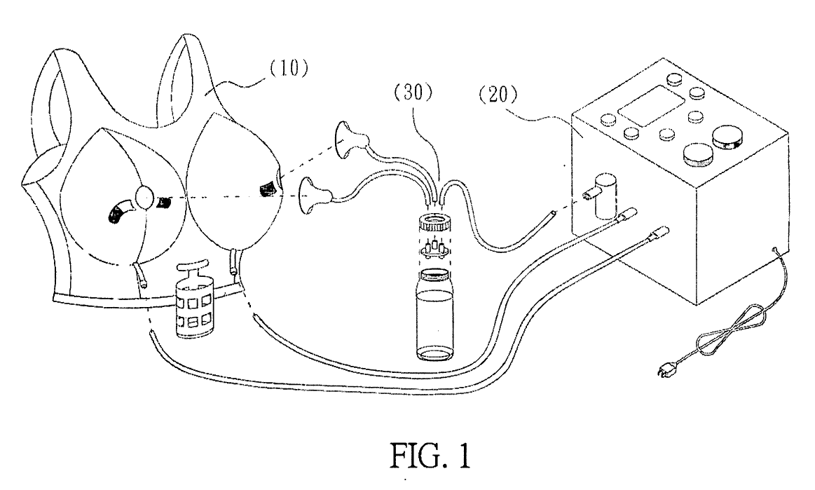

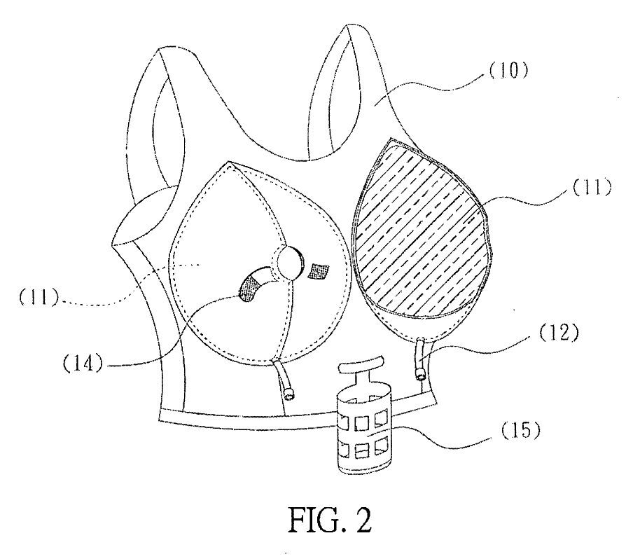

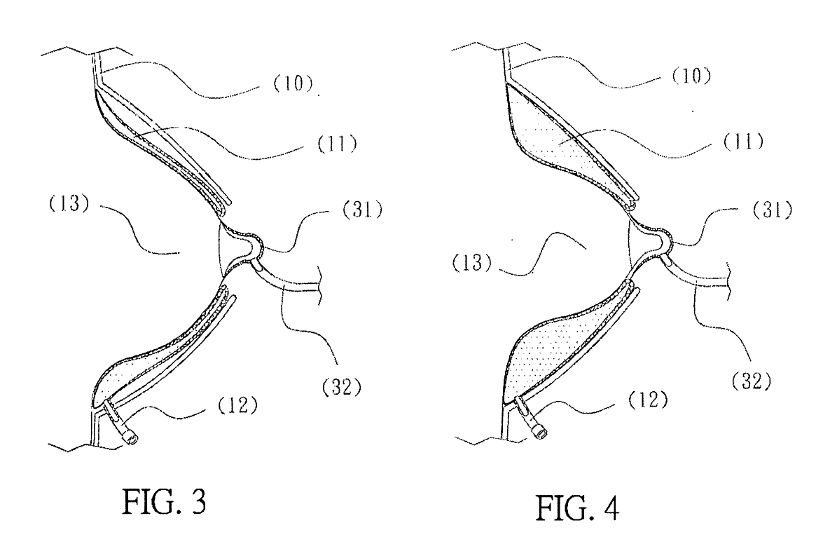

Referring to FIG. 2 and FIG. 3, two cup-shaped air bags is respectively put into two cups of an air bag bra 10 to form two air bag cups 11. The air bag bra 10 has flags for exposing nipples to draining breast milk. A magic pad in the flags is used to fix a milk collection sucking portions 31. Two windpipe connection portions 12 respectively below the two cups are provided to exhaust air from and inhale air into the air bag cups 11. A bottle bag 14 is put at center bottom of the air bag bra 10 for putting baby bottle therein. The film thickness of the air bag cups 11 gradually thickens from bottom to top, and so the air bag cups 11 are expanded first from bottom gradually to top while being inhaled air there into. Therefore, strengths due to the expansion are generated from bottom to top, like as swelling balloon. When the user wore the air bag cups 11 on the breasts, the air bag cups 11 totally squeezes the breasts from bottom of the breasts to nipple for pressing the breast milk lactated by the mammary gland to the nipple. Referring to FIG. 1 and FIG. 3, an air pump 20 generates a negative air pressure to drain the breast milk through milk collection sucking portions 31 and drainage tubes 32 into the baby bottle. The material of the air bag cups 11 may be rubber, silicon rubber, or the like of flexible material. The outer material of the air bag bra 10 may be a stretchless fabric, such as nylon applied to hemadynamometer. Therefore, the strengths can center on the breasts while the air bag is expanding. The inner material of the air bag bra 10 may be a perspiration absorptive and soft material.

Referring to FIG. 5, the basic principle of the air pump 20 is similar to a small-scale reciprocating piston compressor, but the air pump 20 has one more inhaling connector for providing suction to draining the breast milk. The air pump 20 mainly comprises two outputting windpipes 21 of positive pressure warm air, an inhaling connector 22 for negative pressure air, a drain valve 221, an electric heater, mechanical pressure regulator valves 23 and 24, a control circuit, magnetic valve, and an operation panel 25. The drain valve 221 is provided to avoid the breast milk flowing into the air pump 20. The mechanical pressure regulator valves 23 is provided to adjust the pressure of the positive pressure warm air, and the mechanical pressure regulator valves 24 is provided to adjust the pressure of the negative pressure air. Overall, the air pump 20 does not have any special structure and can be bought from market. However, the air pump 20 is controlled by the control circuit and several magnetic valves, and can generate the positive pressure warm air and negative pressure suction that the bar in the present invention needs. The feature of the bra is to provide the user to control the positive air pressure, negative air pressure, exhaust/inhalation period, and air temperature through the operation panel with the control circuit.

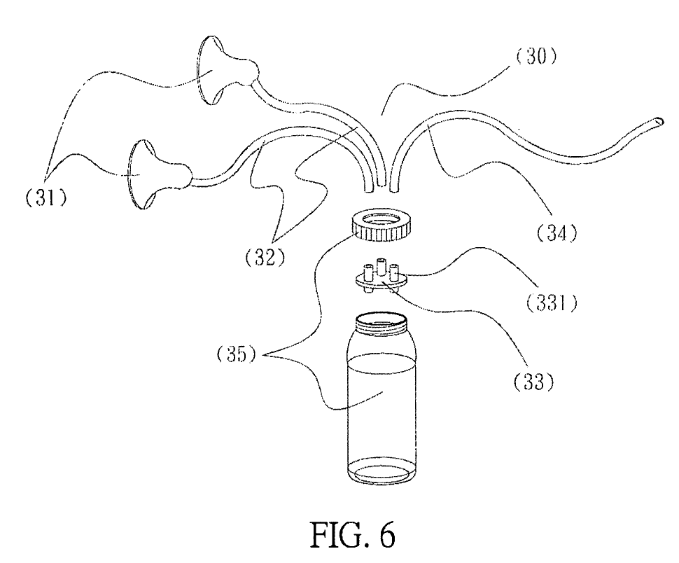

Referring to FIG. 6, a milk drainage assembly 30 is a set of components capable of high temperature disinfection. The milk drainage assembly 30 is made of silicon rubber and poly carbonate resin, and comprises two milk collection sucking portions 31, two drainage tubes 32, a bottle airtight structure 33, and a negative pressure pipe 34 for connecting the bottle airtight structure 33 to the inhaling connector 22. The structure of the bottle airtight structure 33 is very simple and is just like a general baby bottle with a three connector 331 for connecting three pipes. Namely, it is just to take a pacifier of the baby bottle 35 out and lock the bottle airtight structure 33 thereon in the present invention.

Before using the air bag nursing bar of the present invention, the user first has to clean the breasts and then wear an air bag bra 10, as wearing the general bra. The windpipe connection portions 12 of air bag are connected with the outputting windpipes 21 of positive pressure warm air. After that, the milk collection sucking portions 31 is put on the nipples and fixed by the magic pad. The baby bottle 35 is locked with the bottle airtight structure 33. The drainage tubes 32 are connected with the connectors 331 of the bottle airtight structure 33, and the negative pressure pipe 34 is connected a third connector 331 of the bottle airtight structure 33 with the inhaling connector 22 of the air pump 20. Next, the baby bottle 35 is put into the bottle bag 14 of the air bag bra 10. As mentioned above, the preparation for draining breast milk has been finished. The breast milk collection is performed hygienically and comfortably by connecting the air pump 20 to a power source and selecting an automatically sucking process in the operation panel 25.

When the air pump 20 is working, a compressor in the air pump generates required positive and negative pressure airs. The positive pressure air is guided to a mechanical pressure regulator valve 23 that is capable of adjusting air pressure of the positive pressure air. The user can adjust a comfortable maximum expanding pressure of the air bag cups 11 according the user's own breast size. The positive pressure air adjusted by the mechanical pressure regulator valve 23 flows into an electric heater via a windpipe, and then the positive pressure air heated by the electric heater is divided into two windpipes. Each windpipe has a three-way magnetic valve controlled by the control circuit. A reciprocation of the three-way magnetic valve functions as an intake and outlet, and so the positive pressure air becomes a warm pulse positive pressure air with variant air pressure. Furthermore, a set of pressure and temperature sensors after the three-way magnetic valve is electrically connected to the control circuit. Through a monitor of the pressure and temperature sensors, the control circuit changes the reciprocating frequency and the power of the electric heater based on the setup that the user inputted with the operation panel 25. Therefore, the massage strength, period, and temperature can be adjusted as user's hope. Finally, the windpipes is connected to the two outputting windpipes 21 of the air pump 20, and respectively supply warm positive pressure air into left and right air bag cups 11. The negative pressure air is connected to a mechanical pressure regulator valve 24 via a windpipe, and finally the negative pressure air adjusted by the pressure regulator valve 24 flows to the inhaling connector 22 of the air pump 20.

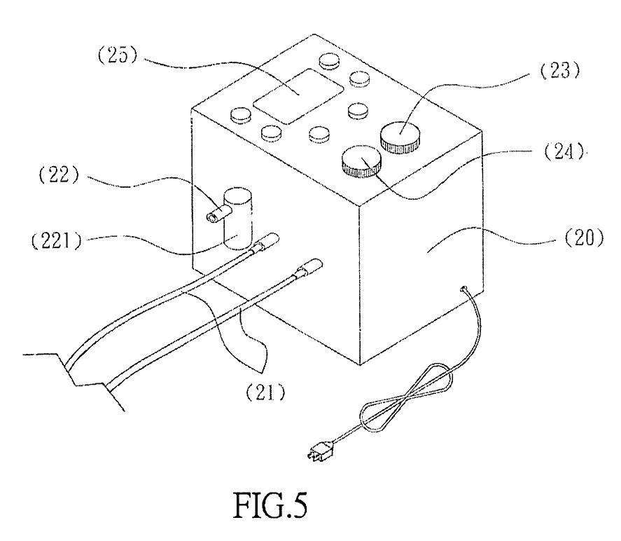

When the automatically sucking process is executing, the air pump 20 first outputs warm pulse positive pressure air, and the air bag cups 11 in the bra rapidly expand and shrink in response to the pulse and so the breast are kneaded as massage. Because the warm air continuously passes in and out, the temperature of the air bag cups 11 raises to warm up the breasts and the mammary gland will lactate. This is the first stage of breast warming and massage. The user can setups a most comfortable massage strength, period, and temperature with the operation panel 25 through the control circuit. When the stage of breast warming and massage is finished in several minutes, the second stage will start. The air pump 20 outputs warm positive pressure air through the pressure regulator valve 23 according to the maximum expanding pressure setup of air bag cups 11. The air bag cups 11 gradually squeeze the total breasts from bottom of the breasts to nipple for draining the breast milk lactated by the mammary gland to the nipple. The drained breast milk is collected in the baby bottle 35 by the milk collection sucking portions 31 and the drainage tubes 32. The user can setup a comfortable suction through the mechanical pressure regulator valve 24 of negative air pressure. Under the control of the control circuit, the air pump 20 keeps the maximum expanding pressure at several seconds and the three-way magnetic valve releases the air in the air bag cups 11 for avoiding the user uncomfortableness. The period action mentioned above executes repeatedly, as shown in FIG. 3 and FIG. 4, until that the breasts don't lactate any more or a breast engorgement is gone. The user presses the stop bottom of the operation panel 25 to finish the milk collection.

The two air bag cups 11 are fed with air individually by the two windpipes, and further controlled by the control circuit and the several valves. Therefore, the user not only setups the massage strength, period, temperature, and pressure for draining milk, but also selects massage two breasts and collecting milk simultaneously or alternatively. Furthermore, the user can perform the breast warming and massage stage or the collecting milk stage individually. Hence, both of the woman after lactation and beauty care can use the air bag nursing bra of the present invention with other medicine material for breast, such as far infrared pad and breast firming cream. Accordingly, the bra of the present invention can be applied as lift bra, contour bra. In conclusion, the bra of the present invention is a multi-function bra with lift, plus size, and breast milk draining.

The essence of the invention for massage and draining is to employ the exhaust (expand) and inhalation (shrink) of the air bag cups and control the temperature, pressure, and pulse frequency of the air. Therefore, the air bag cups are to massage and warm up the breasts or gradually squeezes the total breasts from bottom of the breasts to nipple for draining the breast milk lactated by the mammary gland to the nipple.

Although specific embodiments have been illustrated and described, it will be obvious to those skilled in the art that various modifications may be made without departing from what is intended to be limited solely by the appended claims.

What is claimed is:

1. An air bag nursing bra, comprising:
a bra having two air bag cups, each of said air bag cups having a flag for exposing nipples and connecting with a windpipe extending out of said bra;
a milk drainage assembly comprising two milk collection sucking portions, a bottle airtight structure, two drainage tubes for connecting said bottle airtight structure with said milk collection sucking portions, and a pipe; and
a air pump having an inhaling connector connected to said bottle airtight structure through said pipe, generating a positive pressure air through said windpipe to said air bag cups and a negative pressure air to said milk drainage assembly for draining breast milk to a bottle locked with said bottle airtight structure.

2. The air bag nursing bra according to claim 1, wherein said air bag cups are conoid.

3. The air bag nursing bra according to claim 2, wherein said air bag cups has a film thickness gradually thickening from bottom of conoid to top.

4. The air bag nursing bra according to claim 1, wherein said windpipes are extended form the bottom of said air bag cups.

5. The air bag nursing bra according to claim 1, wherein said air pump has an electric heater to warm up said positive pressure air.

6. The air bag nursing bra according to claim 5, wherein said air pump has a plurality of valves for regulating said warm positive pressure air to a warm pulse positive pressure air.

7. The air bag nursing bra according to claim 6, wherein said air pump has a control circuit electrically connected to said electric heater and said valves for controlling the pressure, temperature, and pulse frequency of said positive pressure air.

8. The air bag nursing bra according to claim 7, wherein air pump has an operation panel electrically coupled to said control circuit for setup said pressure, temperature, and pulse frequency of said positive pressure air.

9. The air bag nursing bra according to claim 1, wherein said air pump has a plurality of valves for regulating said positive pressure air to a pulse positive pressure air.

10. The air bag nursing bra according to claim 1, wherein said milk drainage assembly is made of silicon rubber and poly carbonate resin.
